(19) United States
(12) Patent Application Publication
(10) Pub. No.: US 2023/0293854 A1
(43) Pub. Date: Sep. 21, 2023

(54) Untethered Catheter Containment System and Method

(71) Applicant: Bard Access Systems, Inc., Salt Lake City, UT (US)

(72) Inventors: Christopher Vincent, London (GB); Luke Robbins, Egham (GB); Norihiko Inoue, London (GB); Jim Roberts, Richmond (GB); Kyle G. Thornley, Farmington, UT (US); Joe Spataro, Cottonwood Heights, UT (US); John Stokes, Ogden, UT (US); Glade H. Howell, Draper, UT (US); Lisa I. Bailey, Millcreek, UT (US); Jonathan W. Rutledge, Layton, UT (US); David Brashares, West Jordan, UT (US); Hannah Gracie O'neill, Millcreek, UT (US)

(73) Assignee: Bard Access Systems, Inc., Salt Lake City, UT (US)

(21) Appl. No.: 18/123,843

(22) Filed: Mar. 20, 2023

Related U.S. Application Data
(60) Provisional application No. 63/322,124, filed on Mar. 21, 2022.

(51) Int. Cl. A61M 25/02

(52) U.S. Cl. CPC A61M 25/02; A61M 2025/0183

(57) ABSTRACT

A catheter containment system configured for securing a proximal portion of a catheter placement system within a sterile field. This relieves the clinician from holding the proximal portion to prevent them for falling outside of the sterile field. Further, the proximal portion can be secured out of the way from the insertion site providing a clear field of view. Embodiments include a body, having a channel configured to releasably engage an extension leg or guidewire, and a securement feature to secure the body to a portion of a drape or similar item of the sterile field. Exemplary securement features can include clips, clasps, pins, magnetic elements, adhesives, twist ties, zip ties, snap fasteners, hook and loop attachments, mechanical grasping devices or adhesive pads. The system can further include anchor tabs that can be adhered to the drape to provide an anchor point.

### PRIORITY

This application claims the benefit of priority to U.S. Provisional Patent Application No. 63/322,124, filed Mar. 21, 2022, which is incorporated by reference in its entirety into this application.

### BACKGROUND

Central venous catheters (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma and/or increased risk of infection. There is a relatively high potential for touch contamination due to the number of medical devices that need to be interchanged during the Seldinger technique. As such, advanced catheter placement systems have been developed to reduce the number of steps and medical devices involved in placing a catheter, such as a CVC, into a patient.

Exemplary advanced catheter placement systems can include, for example, rapidly insertable central catheter (RICC) placement systems. The RICC includes a monoluminal access section disposed distally and having a first diameter, a multi luminal catheter section disposed proximally and having a second diameter larger than then first diameter, and a tapered dilation section disposed therebetween and extending between the first diameter and the second diameter.

Whether using a traditional Seldinger techniques or using RICC placement systems, often the clinician is required to stabilize a proximal portion of the catheter placement system, (e.g. hub, extension legs, guidewires, etc.) while placing a distal tip of the catheter within the vasculature. This prevents these proximal portions of the catheter placement system from tangling or falling outside of the sterile field. However, holding these proximal portions can reduce the dexterity of the clinician and/or become entangled, complicating the procedure. Further these proximal portions are at risk from falling outside of the sterile field, contaminating the device and causing the clinician to abort the placement procedure. What is needed therefore is a catheter containment system that allows a clinician to secure the proximal portions of the catheter placement system during placement, without reducing the dexterity of the clinician and mitigating these proximal portions from falling outside of the sterile field. Embodiments disclosed herein are directed to address at least the foregoing.

### SUMMARY

Disclosed herein is a catheter containment system for retaining a portion of a catheter placement system within a sterile field including, a containment device including, a body defining a channel extending longitudinally along a first axis, the channel configured to retain the portion of the catheter placement system, and having an opening extending longitudinally and configured to allow ingress or egress along a second axis extending at an angle to the first axis of the channel, and a securement feature coupled to the body and configured to releasably engage a portion of the sterile field proximate an insertion site.

In some embodiments, the portion of the catheter placement system includes one of a catheter body, catheter hub, extension leg, luer lock, or guidewire.

In some embodiments, the body includes a first channel configured to retain a first extension leg and a second channel configured to retain a second extension leg.

In some embodiments, the body includes a first channel configured to retain a first extension leg and a second extension leg.

In some embodiments, the catheter placement system includes a RICC catheter placement system for placing a rapidly insertable central catheter.

In some embodiments, the body is rotatably, hingedly or pivotably coupled to the securement feature.

In some embodiments, the securement feature includes one of a spring clip, butterfly clip, beret clip, alligator clip, clothes pin, safety pin, or suction cup.

In some embodiments, the catheter containment system further includes an anchor having a foot plate and a tab extending at an angle thereto, the foot plate include an adhesive layer and the tab configured to be engaged by the securement feature.

In some embodiments, wherein the securement feature includes a first clip member hingedly coupled to a second clip member, the first clip member including a first gripping feature configured to engage a second gripping feature of the second clip member to secure the containment device to the portion of the sterile field.

In some embodiments, wherein the securement feature further includes a biasing member configured to bias the securement feature to a closed position, the biasing member including one of a spring, compression spring, leaf spring, O-ring, rubber grommet, or magnetic element.

In some embodiments, the catheter containment system further includes a base coupled to both the body and the securement feature, and wherein the securement feature includes a first coupling disposed on the base, and a second coupling disposed on the portion of the sterile field.

In some embodiments, the securement feature includes one of a hook and loop fastener, snap fastener, a magnetic element, or adhesive.

In some embodiments, the securement feature includes a tie selected from a group consisting of a string, cord, wire, twist tie, or zip tie.

In some embodiments, the securement feature includes a hook slidably engaged with a sleeve, the hook having an arm with a finger plate disposed at a first end of the arm and a J-hook disposed at a second end of the arm, opposite the finger plate, the arm configured to receive a portion of the sterile field between the J-hook and the sleeve.

In some embodiments, the securement feature includes a sleeve and two or more hooks slidably engaged therewith, each of the two or more hooks slidable between a retracted position, open position, and a closed position.

Also disclosed is a catheter placement and containment system for placing a catheter within a vasculature of a patient including, a catheter placement device having, a needle, a catheter including an extension leg, and a guidewire, a sterile drape configured to define a sterile field around the insertion site, and a catheter containment system having, a body defining a channel and configured to engage a portion of the catheter placement device, and a securement feature coupled to the body and configured to secure the catheter containment system to the sterile drape.

In some embodiments, the securement feature includes one of a spring clip, butterfly clip, beret clip, alligator clip, clothes pin, safety pin, or suction cup.

In some embodiments, the system further includes an anchor having a foot plate and a tab extending at an angle thereto, the foot plate include an adhesive layer and the tab configured to be engaged by the securement feature.

In some embodiments, the system further includes a base coupled to both the body and the securement feature, and wherein the securement feature includes a first coupling disposed on the base, and a second coupling disposed on the sterile drape.

In some embodiments, the securement feature includes one of a hook and loop fastener, snap fastener, a magnetic element, or adhesive.

Also disclosed is a method of placing a catheter including, providing a catheter placement system including a needle, a catheter, and a guidewire, releasably engaging a portion of the catheter placement system with the catheter containment device, securing the catheter containment device with a portion of a sterile field, and placing the catheter within a vasculature through an insertion site within the sterile field.

In some embodiments, the method further includes adhering the portion of the catheter placement system to a top surface of the catheter containment device, the catheter containment device including a flexible pad coupled to the portion of the sterile field.

In some embodiments, the catheter containment device includes one of a spring clip, butterfly clip, beret clip, alligator clip, clothes pin, safety pin, suction cup, string, cord, wire, twist tie, or zip tie.

Also disclosed is a catheter containment device for retaining a portion of a catheter placement system including, a body defining a channel extending along a longitudinal axis, the channel configured to permanently retain the portion of the catheter placement system, and a handle coupled to the body and configured to releasably engage a portion of a sterile field proximate an insertion site.

In some embodiments, the portion of the catheter placement system includes a proximal end of a guidewire permanently affixed to the body.

In some embodiments, the portion of the catheter placement system includes a proximal portion of a guidewire slidably engaged with the body.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a catheter placement system in an unfolded configuration, in accordance with embodiments disclosed herein.

FIG. 1B shows a plan view of a catheter placement system in a folded configuration ready for use, in accordance with embodiments disclosed herein.

FIG. 2 shows a side view of a catheter of the catheter placement system of FIG. 1A in an unfolded configuration, in accordance with embodiments disclosed herein.

FIG. 3A shows close up detail of a distal portion of the catheter of FIG. 2, in accordance with embodiments disclosed herein.

FIGS. 3B-3C show cross-section views of the catheter of FIG. 3A, in accordance with embodiments disclosed herein.

FIG. 4A shows a perspective view of a spring clip catheter containment system, in accordance with embodiments disclosed herein.

FIG. 4B shows the catheter containment system of FIG. 4A in an exemplary environment of use, in accordance with embodiments disclosed herein.

FIG. 4C shows close up detail of the catheter containment system of FIG. 4A, in accordance with embodiments disclosed herein.

FIG. 5 shows a proximal portion of an exemplary catheter placement system, in accordance with embodiments disclosed herein.

FIGS. 6A-6C show perspective views of catheter containment systems, in accordance with embodiments disclosed herein.

FIG. 6D shows an exemplary method of use for a catheter containment system, in accordance with embodiments disclosed herein.

FIG. 7A shows a perspective view of an anchor of a catheter containment system, in accordance with embodiments disclosed herein.

FIG. 7B shows a perspective view of an anchor in a first configuration, in accordance with embodiments disclosed herein.

FIG. 7C shows a perspective view of an anchor in a second configuration, in accordance with embodiments disclosed herein.

FIG. 7D shows a catheter containment system including an anchor in an exemplary environment of use, in accordance with embodiments disclosed herein.

FIGS. 8A-8B show a catheter containment system, in accordance with embodiments disclosed herein.

FIG. 8C shows close up detail of a catheter containment system including a releasable coupling, in accordance with embodiments disclosed herein.

FIG. 8D shows close up detail of a catheter containment system including a tie, in accordance with embodiments disclosed herein.

FIG. 8E shows a proximal portion of a catheter placement system including a catheter containment system coupled thereto, in accordance with embodiments disclosed herein.

FIG. 9A shows a perspective view of a catheter containment system, in accordance with embodiments disclosed herein.

FIG. 9B shows a cross-sectional view of a catheter containment system, in accordance with embodiments disclosed herein.

FIGS. 9C-9E show an exemplary method of use for a catheter containment system, in accordance with embodiments disclosed herein.

FIGS. 10A-10C show an exemplary method of use for a catheter containment system, in accordance with embodiments disclosed herein.

FIG. 11 shows a proximal portion of a catheter placement system including a catheter containment system coupled thereto, in accordance with embodiments disclosed herein.

FIG. 12A shows a catheter containment system in an exemplary environment of use, in accordance with embodiments disclosed herein.

FIG. 12B shows a side view of the catheter containment system of FIG. 12A, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

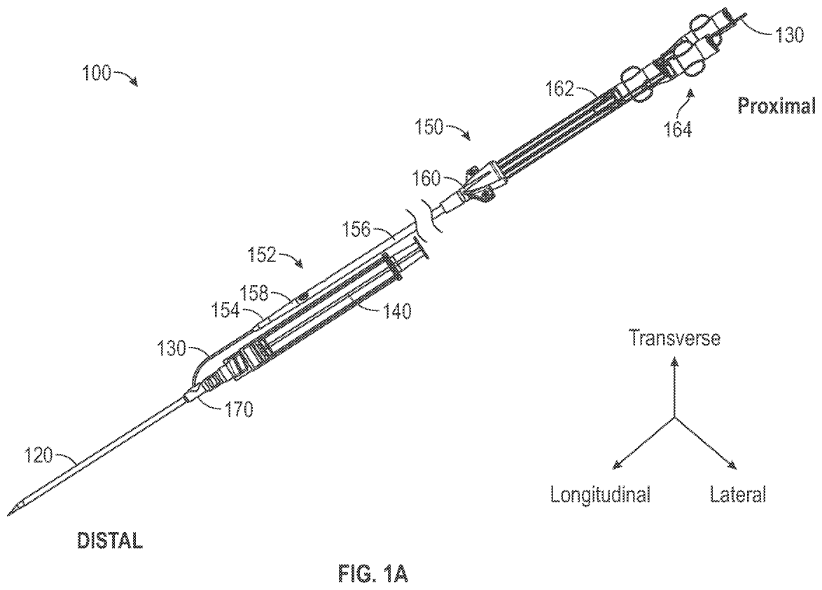

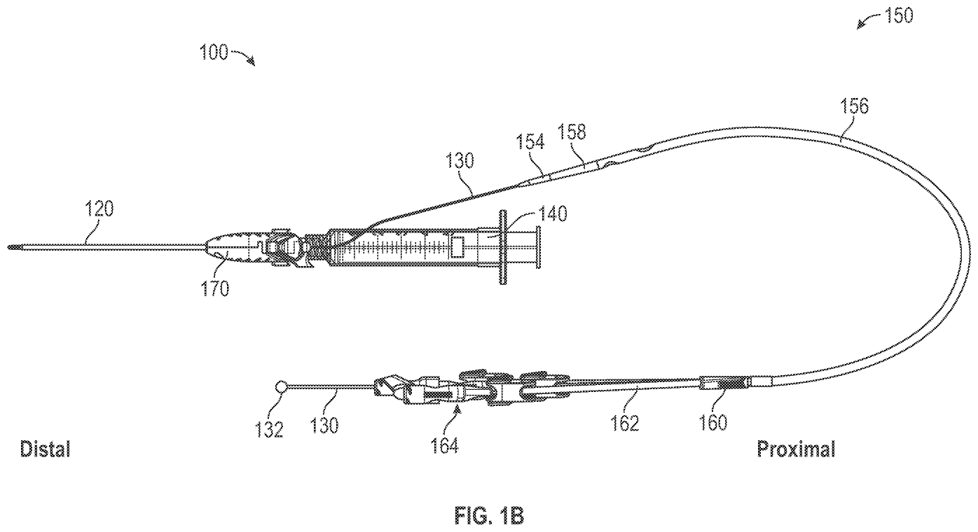

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes. A horizontal plane can be defined by the lateral axis and the longitudinal axis. A vertical plane extends normal to the horizontal plane.

Unless defined otherwise, all technical and scientific terms used herein have the same meaning as commonly understood by those of ordinary skill in the art.

FIGS. 1A-1B show an exemplary catheter placement system (“system”) 100, generally including a needle 120, a guidewire 130, a blood flash system 140, a catheter 150, and a needle housing (“housing”) 170. FIG. 1A shows the system 100 in an unfolded configuration for ease of illustration. FIG. 1B shows a plan view of the system 100 in a folded configuration ready for use. In an embodiment, the catheter placement system 100 can be a Rapidly Insertable Central Catheter (RICC) placement system 100 configured to place a RICC 150. However, it will be appreciated that other catheter placement systems configured to place other types of catheters are also contemplated. Exemplary catheters 150 also include peripheral intravenous (PIV) catheters, peripherally inserted central catheter (PICC), central venous catheters (CVC), midline catheters, dialysis catheters, single lumen catheters, multi-lumen catheters, or the like.

In an embodiment, the catheter 150 generally includes a catheter body 152 supported at a proximal end by a catheter hub (“hub”) 160. The hub 160 includes one or more extension legs 162 extending proximally therefrom and includes a fluid coupling device, such as a luer lock 164, disposed at a proximal end of one of the one or more extension legs 162. The luer lock 164 is configured to couple the extension leg with a medical fluid line, syringe, or the like. Each extension leg of the one or more extension legs 162 is in fluid communication with a lumen of the catheter body 152. For example, a first extension leg 162A is in fluid communication with a proximal lumen 114A, a second extension leg 162B is in fluid communication with a medial lumen 114B, and a third extension leg 162C is in fluid communication with a distal lumen 114C. The catheter body 152 includes an access section 154 disposed distally, a catheter section 156 disposed proximally, and a dilation section 158 disposed therebetween. The access section 154 defines a single lumen and has a first outer diameter, the catheter section 156 defines two or more lumen and has a second diameter larger than the first diameter. The dilation section 158, disposed between the access section 154 and the catheter section 156, defines a tapered outer profile extending from the first diameter of the access section 154 to the second diameter of the catheter section 156. A guidewire 130 can extend through a lumen of the catheter 150 from a proximal end of an extension leg 162, to a distal tip of the access section 154.

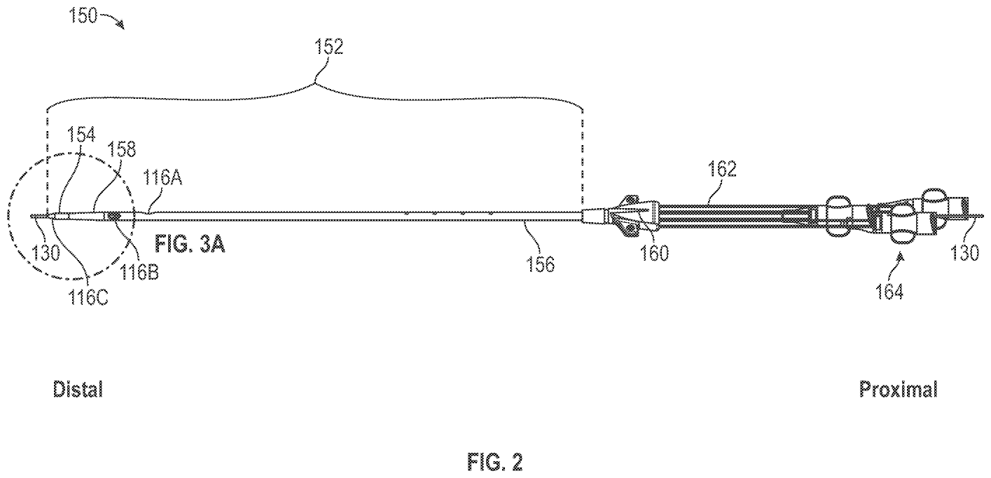

FIG. 2 shows further details of an exemplary catheter 150 of the system 100. In an embodiment, different sections of the catheter 150 are required to perform different functions and as such are required to display different mechanical properties. For example, the access section 154 and the dilation section 158 can provide a more rigid mechanical properties or harder durometer material relative to the catheter section 156. As such, the access section 154 and dilation section 158 can withstand greater axial forces without kinking or collapsing, as these sections are urged distally, forming and dilating the insertion site 50. The catheter section 156 can be formed of a softer durometer, or a more compliant material to facilitate negotiating the catheter section 156 through tortuous vascular pathways.

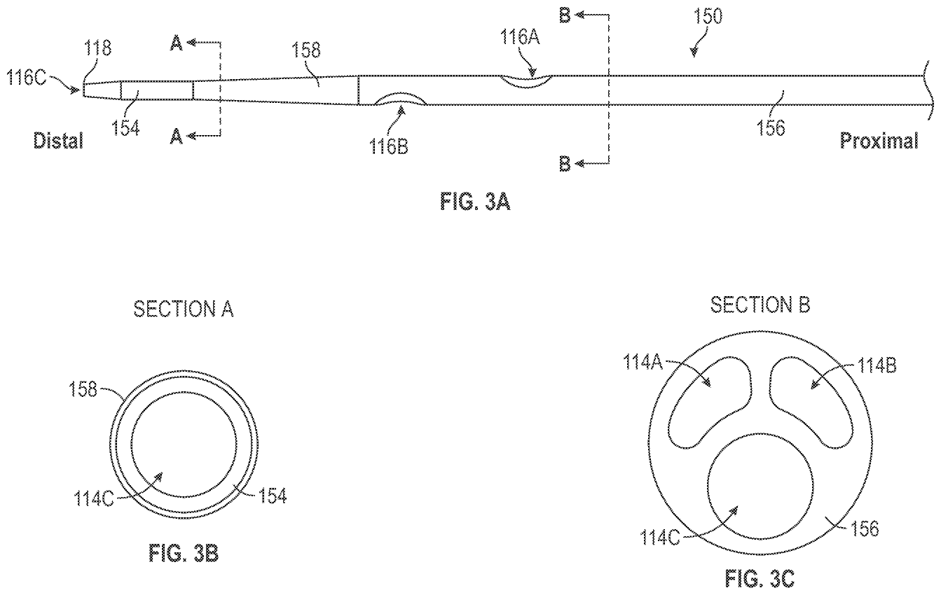

FIGS. 3A-3C show further details of a distal portion of the catheter 150, including the access section 154, the catheter section 156, and the dilation section 158. In an embodiment, the catheter section 156 includes a proximal lumen 114A terminating at a proximal lumen aperture 116A, and a medial lumen 114B terminating at a medial lumen aperture 116B. In an embodiment, each of the proximal lumen aperture 116A and the medial lumen aperture 116B extends through a side wall of the catheter section 156. In an embodiment, each of the proximal lumen aperture 116A and the medial lumen aperture 116B are disposed proximally of the dilation section 158. In an embodiment, the proximal lumen aperture 116A can be disposed proximally of the medial lumen aperture 116B. In an embodiment, the proximal lumen aperture 116A and the medial lumen aperture 116B can be disposed equidistant from a distal tip of the catheter 150.

FIG. 3B shows a cross section view of the catheter body 152 at point “A” of FIG. 3A. As shown, the access section 154 defines a single lumen and a relatively smaller outer diameter. In an embodiment, a proximal portion of the access section 154 can be received within a distal portion of the dilation section 158. A distal lumen 114C of the catheter 150 can extend to a distal tip 118 of the catheter 150 and communicates with a distal lumen aperture 116C. FIG. 3C shows a cross section view of the catheter section 156 at point “B” of FIG. 3A, showing the proximal lumen 114A, medial lumen 114B and distal lumen 114C.

Further details and embodiments of such catheter placement systems 100 can be found, for example, in U.S. Pat. Nos. 11,517,719, 10,376,675, U.S. 2019/0255294, U.S. 2021/0069471, U.S. 2021/0113809, U.S. 2021/0113810, U.S. 2021/0121667, U. S. 2021/0121661, U. S. 2021/0228843, U. S. 2021/0283381, U. S. 2021/0322729, U.S. 2021/0361915, U.S. 2021/0330941, U.S. 2021/0330942, U.S. 2021/0402149, U.S. 2022/0001138, U.S. 2021/0402142, U.S. 2022/0032013, U.S. 2021/0402153, U. S. 2021/0379336, U. S. 2021/0283368, U. S. 2022/0062528, U. S. 2022/0032014, U. S. 2022/0126064, U. S. 2022/0193378, U. S. 2022/0176081, U. S. 2022/0193376, U. S. 2022/0193377, U. S. 2022/0152368, U. S. 2022/0176082, U. S. 2022/0193379, U.S. 2022/0296862, U.S. 2022/0323723, U.S. 2022/0370762, U.S. 2022/0362524, U.S. 2023/0043989, U.S. 2023/0041261, U.S. 2023/0039733, U.S. 2023/0042898, each of which is incorporated by reference in its entirety into this application.

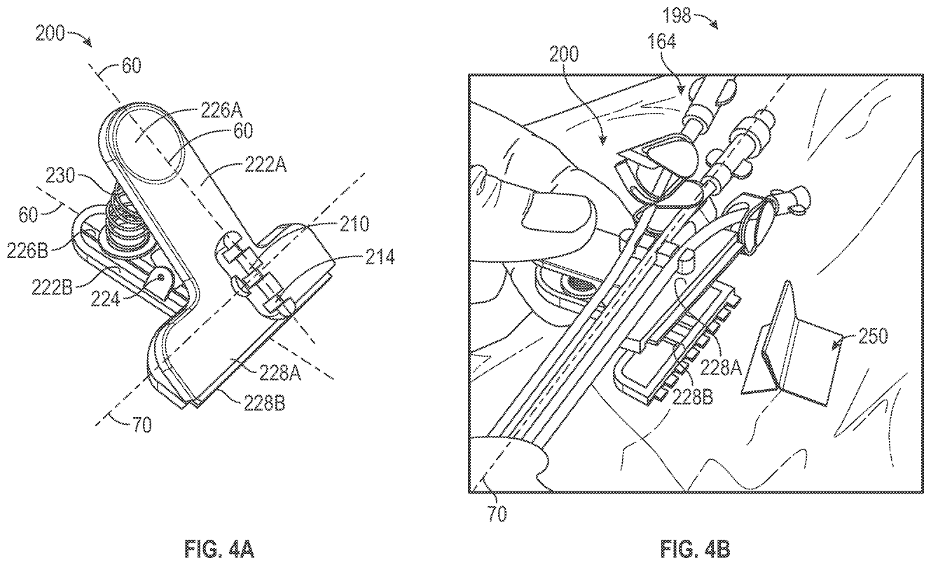

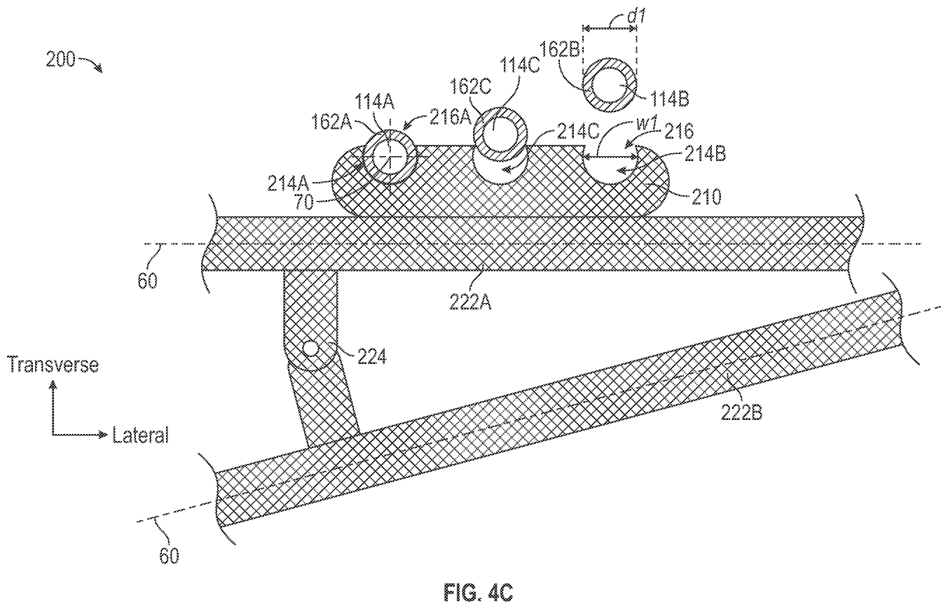

FIG. 4A shows a spring-clip, catheter containment device (“device”) 200. FIG. 4B shows a spring clip catheter containment device 200 in an exemplary environment of use. FIG. 4C shows close up detail of the catheter containment device 200 of FIG. 4A. The device 200 generally includes a body 210 defining one or more channels 214 and a securement feature, such as a spring clip (“clip”) 220 coupled thereto. The body 210 is configured to retain a portion of the catheter placement system 100, for example a portion of the catheter body 152, hub 160, one or more extension legs 162, luer lock 164, guidewire 130, combinations thereof, or the like.

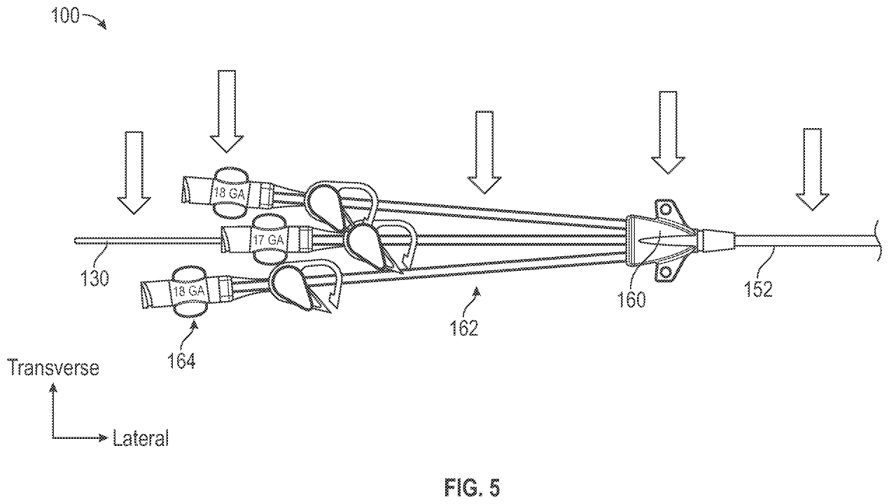

FIG. 5 shows some exemplary portions of the catheter placement system 100 that can be retained by the device 200. However, it will be appreciated that this is not intended to be limiting in any way. Advantageously, securing one or more portions of the catheter placement system 100 together, e.g. one or more extension legs 162, facilitates speed of placement, keeping portions of the catheter placement system 100 organized and mitigating the portions getting tangled. Further, the device 200 coupled with the proximal portions of the system 100 mitigates these portions from falling outside of the sterile field, preventing contamination thereof, as discussed in more detail herein.

In an embodiment, one or more of the proximal portions of the catheter placement system 100, e.g. extension legs 162, guidewire 130, etc., can be releasably retained by the body 210, as described in more detail herein. In an embodiment, one or more of the proximal portions of the catheter placement system 100 can be permanently and/or slidably engaged with the device 200, e.g. one of the body 210 or the securement feature 220, preventing the system 100 from disengaging from the device 200 and accidentally falling outside of the sterile field. In an embodiment, one or more of the proximal portions of the catheter placement system 100 can be permanently affixed to the device 200, e.g. one of the body 210 or the securement feature 220. For example, a proximal end of the guidewire 130 can be formed integrally with, or permanently coupled to, the body 210. Advantageously, this can prevent the guidewire 130 from also be drawn through the catheter placement system 100 and into the vasculature, as well as preventing the guidewire 130 from falling outside of the sterile field.

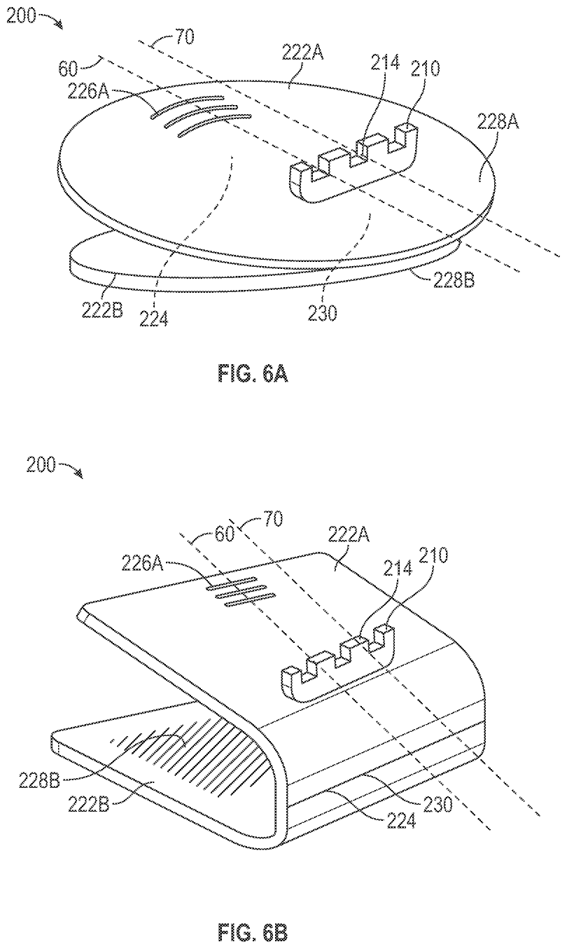

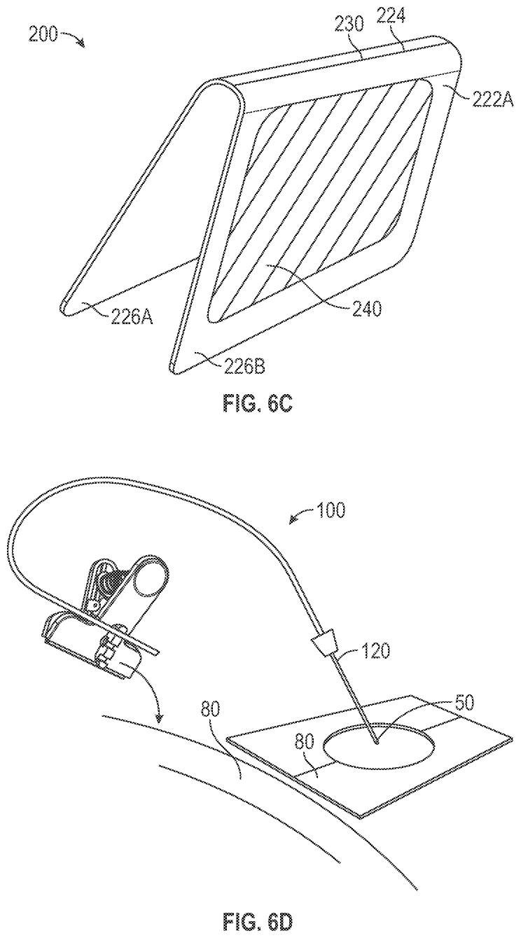

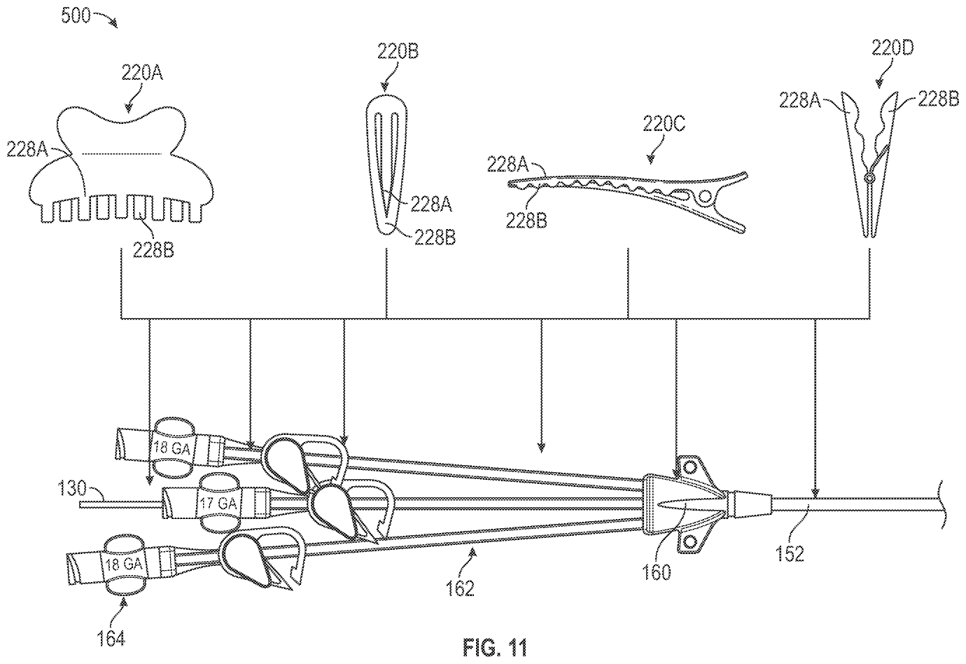

In an embodiment, the securement feature, i.e. spring clip 220, is configured to releasably secure the device 200 to a portion of the sterile field 80, surrounding an insertion site 50. Exemplary portions of the sterile field 80 include, but are not limited to, an article of clothing on the patient, a patient surgical gown, sleeve, dressing gown, a medical device coupled to the patient such as an adhesive dressing, anchor pad, an article of clothing on the clinician, surgical gown, scrubs, sleeve, glove, bed linen, surgical drape, operating table drape, combinations thereof, or the like. In an embodiment, one or both of the body 210 and the spring clip 220 can be formed of a substantially rigid, or resilient material such as a plastic, polymer, metal, alloy, composite, or the like. In an embodiment, the securement feature 220 can include a butterfly clip 220A, beret clip 220B, alligator clip 220C, clothes pin 220D, safety pin, suction cup and can be coupled to the body 210, as described herein. Exemplary securement features 220 are shown in FIG. 11. As such, the securement feature 220 can be configured to secure the device 200 to various portions of the sterile field 80 depending on the shape, size, and material of the sterile field to be secured to. Advantageously, as shown in FIG. 6D, the device 200 can retain the proximal portions of the catheter placement system 100 away from the insertion site 50, providing improved visibility, a simplified presentation, and greater control.

With continued reference to FIG. 4A, in an embodiment, the spring clip 220 includes a first clip member 222A hingedly coupled to a second clip member 222B by a hinge 224. Each of the first clip member 222A and the second clip member 222B extend along an axis 60 between a first end and a second end, the second end being opposite the first end. The first end of the clip member 222 includes a handle 226. The second end of the clip member 222, opposite the first end, includes a gripping feature 228. The hinge 224 can be disposed between the first end and the second end of the clip member 222 and is configured to allow the first clip member 222A to pivot relative to the second clip member 222B between an open position and a closed position.

In the open position, as shown in FIG. 4B, the first gripping feature 228A disposed on the first clip member 222A is disposed in a spaced apart relationship relative to the second gripping feature 228B. In the closed position, as shown in FIG. 4A, the first gripping feature 228A disposed on the first clip member 222A is disposed adjacent to, or contacting, the second gripping feature 228B. In an embodiment, the gripping feature(s) 228 include interlocking teeth, lips, flanges, or can include a second material different from a first material of the device 200 that has a relatively high frictional coefficient, such as silicone rubber, elastomer, adhesive or the like. As such, the gripping feature 228 provides increased friction between the spring clip 220 and the portion of the sterile field 80 disposed between the gripping features 228.

The spring clip device 200 further includes a biasing member 230 configured to bias the spring clip 220 towards the closed position. Exemplary biasing members 230 include, but are not limited to, springs, compression springs, leaf springs, O-rings, rubber grommets, or similar devices formed from plastic, polymer, elastomer, metal, alloy, Nitinol, composites or the like and configured to elastically deform. As shown, the biasing member 230 is a compression spring and is disposed between the hinge 224 and the handle 226 to urge the device 200 towards the closed position.

In an embodiment, for example as shown in FIG. 6A, the biasing member 230 can be a magnetic element and disposed along the clip member axis 60 between the hinge 224 and the gripping feature 228 and configured to urge the device 200 towards the closed position. The device 200 can include a first magnetic element disposed on a first clip member 222A and a second magnetic element disposed on the second clip member 222B and configured to attract the first magnetic element towards the second magnetic element. In an embodiment, the magnetic element biasing member 230 can be disposed between the hinge 224 and the handle 226 and configured to urge the device 200 towards the closed position. The device 200 can include a first magnetic element disposed on a first clip member 222A and a second magnetic element disposed on the second clip member 222B and configured to repel the first magnetic element away from the second magnetic element. As used herein, a magnetic element can include a permanent magnet, electro-magnet, a magnetized ferrous material, or a ferrous material.

FIG. 4C shows further details the device 200. In an embodiment, the body 210 can be coupled to one of the first clip member 222A or the second clip member 222B. In an embodiment, the body 210 can include one or more channels 214 formed therein and extending longitudinally along a first axis 70. The channel 214 can be configured to releasably retain a portion of the catheter placement system 100. For example, as shown in FIG. 4C, the body 210 includes a first channel 214A configured to receive a first extension leg 162A, a second channel 214B configured to receive a second extension leg 162B, and a third channel 214C configured to receive a third extension leg 162C. However, this is not intended to be limiting and other portions of the catheter placement system 100, e.g. luer lock 164, hub 160, extension legs 162, guidewire 130, etc., can be received by each of the first channel 214A, second channel 214B, or third channel 214C to be releasably retained therein.

In an embodiment, a portion of the catheter placement system 100 is permanently affixed to the body 210 or permanently retained within a channel 214 of the body 210. For example, the body 210 may include a channel 214 that does not have an opening 216 and instead encircles the extension leg 162, guidewire 130, etc. completely. As such, the body 210 can slidably engage the portion of the catheter placement system 100 but cannot be detached therefrom. To note, the guidewire 130 can include a plug 132 permanently affixed to a proximal end thereof, similarly the extension leg 162 can include a luer lock 164 or the like attached to the proximal end thereof. As such, the plug 132 or luer lock 164 can abut against the body 210 to prevent the guidewire 130 or extension leg 162 from slidably disengaging the body 210 along the axis of the channel 214.

In an embodiment, a proximal end of the guidewire 130, and/or plug 132, can be permanently affixed to the device 200. For example, the plug 132 can be hingedly, pivotably, or rotatably coupled to the body 210 or the securement feature 220. Alternatively, the plug 132, or a proximal portion of the guidewire 130, can be attached to or formed integrally with the device 200, e.g. by adhesive, bonding, welding, ultrasonic welding, or the like.

Advantageously, permanently affixing a proximal portion of the catheter placement system 100 to the device 200 prevents the catheter placement system 100 from accidently disengaging the device 200 and falling outside of the sterile field. Further, the device 200 functions as a plug 132 to prevent a guidewire 130, for example, from being accidentally drawn through the catheter lumen and into the vasculature.

In an embodiment, the channel 214 includes an opening 216, extending axially with the channel 214 and configured to allow ingress or egress of the portion of the catheter 150 to/from the channel 214. In an embodiment, a width (w1) of the opening 216 can be less than a diameter (d1) of the portion of the catheter 150 to be retained by the channel 214. As such, one or both of the extension leg 162 and the channel opening 216 must elastically deform slightly to receive the extension leg 162 within the channel 214. Further the opening 216 can abut against the extension leg 162 to retain the extension leg 162 within the channel 214. As such, the channel 214 can retain the extension leg 162 in a snap fit engagement. In an embodiment, a width of the channel 214 can be equal to or smaller than an outer diameter of the portion of the catheter 150 and can engage the portion of the catheter 150 in an interference fit, press-fit, or snap fit engagement to releasably retain the portion of the catheter 150 therein.

In an exemplary method of use, a clinician can couple the device 200 to a portion of the catheter placement system 100. For example, a first extension leg 162A can be urged through a first opening 216A and into the first channel 214A, a second extension leg 162B can be urged through a second opening 216B and into the first channel 214B, and a third extension leg 162C can be urged through a third opening 216C and into the third channel 214C. Advantageously, the device 200 can retain the extension legs 162, preventing them becoming tangled as well as preventing them from falling outside of the sterile field. As will be appreciated, other portions of the catheter placement system 100, such as a portion of the guidewire 130, luer lock 164, extension leg 162, catheter hub 160, catheter body 152, combinations thereof, or the like can also be retained, without limitation.

The clinician can then urge a first handle 226A of the first clip member 222A towards the second handle 226B of the second clip member 222B, overcoming the force of the biasing member 230 and pivoting the first gripping feature 228A away from the second gripping feature 228B to transition the device 200 to the open position (FIG. 4B). The clinician can then pinch a portion of the sterile field 80 (FIG. 6D) and can then place the portion of the sterile field 80 between the first gripping feature 228A and the second gripping feature 228B and release the handles 226 to allow the biasing member 230 to urge the device 200 towards the closed position (FIG. 4A), securing the device 200 and the portion of the catheter placement system 100 coupled thereto, within the sterile field 80.

As shown in FIGS. 4A-4C, the axis 70 of the channel 214 can be aligned perpendicular to an axis 60 of the clip member 222. However, it will be appreciated that the axis 70 of channel(s) 214 can be aligned parallel to, or at an angle to, an axis 60 of the clip member 222. For example, as shown in FIG. 6A, the axis 70 of the channel 214 can be aligned parallel to an axis of the clip member 222. In an embodiment the body 210 can be rotatably coupled to the securement feature 220 to allow an axis 70 of the channel 214 to rotate or pivot relative to the axis 60 of the securement feature 220. In an embodiment, the body 210 can be releasably coupled to the spring clip 220 using an interference fit, press-fit, snap-fit engagement, a latching mechanism, adhesive, a frangible bridge, hook and loop attachments, combinations thereof, or the like.

FIG. 5 shows a proximal portion of the catheter placement system 100 and indicates various structures of the catheter placement system 100 that can be retained by the channel 214. Exemplary structures of the system 100 can include a portion of the guidewire 130, luer lock 164, extension leg 162, catheter hub 160, catheter body 152, combinations thereof, or the like.

In an embodiment, the shape of the clip member 222 can vary. As shown in FIG. 4A, the clip member 22 can substantially define a “T”-shape piece, wherein a width of the gripping feature 228 is wider than a width of the handle 226. As shown in FIG. 6A, the clip member 222 can substantially define a circular, or disc shape. As shown in FIG. 6B, the clip member 222 can substantially define a rectangular shape. It will be appreciated that other regular or irregular three-dimensional shaped clip members are also contemplated to fall within the scope of the present invention, such as circular, oval, ellipsoidal, polygonal, or the like.

In an embodiment, the relative positions of the hinge 224, handle 226, gripping feature 228, and biasing member 230 can vary along the axis 60 of the clip member 222. For example, FIG. 6B shows an embodiment of the device 200 including the handle 226 disposed at the first end and the hinge 224 disposed at the second end. The gripping feature 228 can be disposed on an inside surface of the clip member 222 at a point between the first end and the second end. In an embodiment, the biasing member 230 can be disposed at the second end, for example the spring can be incorporated with the hinge 224 mechanism. These and other combinations of the hinge 224, handle 226, gripping feature 228, and biasing member 230 positioned on the clip member 222 are also contemplated to fall within the scope of the present invention.

In an embodiment, as shown in FIG. 6C, the device 200 can include a “non-slip” pad 240 disposed on a surface of the device 200, e.g. a surface of the securement feature 220. In an embodiment, the non-slip pad 240 can include a material having a relatively high frictional co-efficient such as rubber, silicone rubber, polymer, elastomer, or the like. In an embodiment, the non-slip pad 240 can include a light-tack adhesive layer. Advantageously, the non-slip pad 240 allows the device 200, including the portion of the catheter placement system 100 coupled therewith, to be placed on to a portion of the sterile field 80 (e.g. a drape, etc.) and to mitigate the device 200 and catheter placement system 100 assemble from slipping or falling outside of the sterile field 80 without having to clip the device 200 to the drape 80. The non-slip pad 240 can be repositioned a number of times whilst maintaining its non-slip functionality.

FIG. 6D shows an exemplary method for attaching the catheter containment device 200 to a portion of the sterile field 80, e.g. a drape. This can include a clinician manually pinching the drape 80 to bunch the material and thereby providing enough material for the catheter containment device 200 to grasp, as described herein.

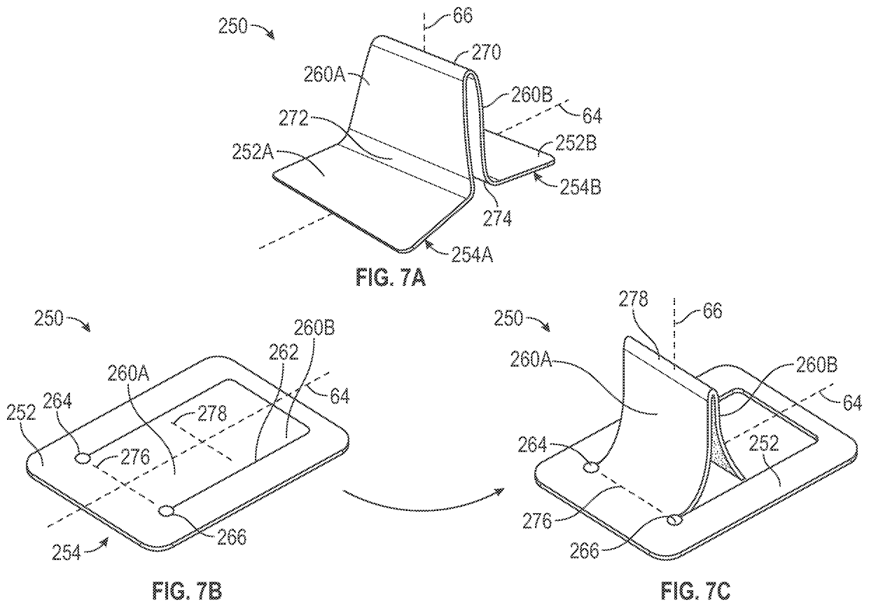

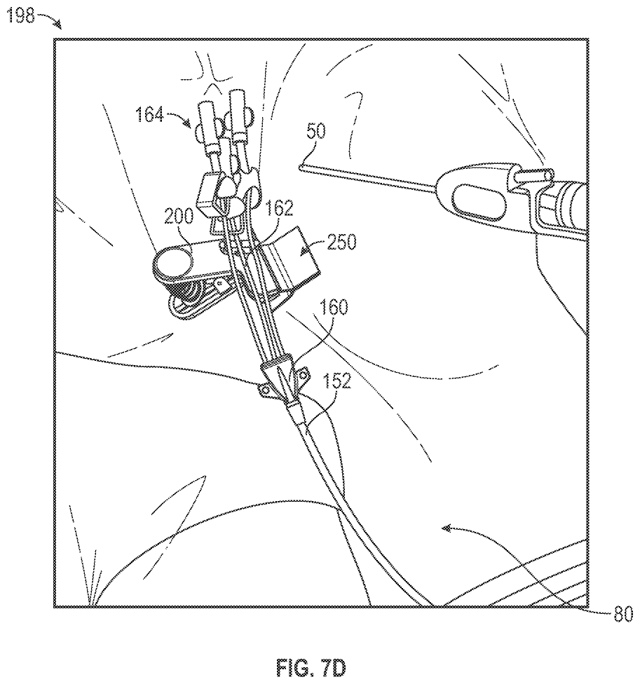

In an embodiment, a catheter containment system 198 can include the catheter containment device 200, and can further include an anchor device (“anchor”) 250. FIGS. 7A-7F show further details of exemplary anchors 250. The anchor 250 can generally include a foot plate 252 and a tab 260.

The foot plate 252 can include an adhesive layer disposed on a first surface 254 of the foot plate 252, for example a lower surface of the foot plate 252, and is configured to attach the anchor 250 to a surface of the sterile field 80. The tab 260 can extend from the foot plate 252, at an angle thereto, and can be configured to engage the clip 220. For example, the tab 260 can extend between the first gripping feature 228A and the second gripping feature 228B and can be secured therebetween when the device 200 is in the closed position. Advantageously, the anchor 250 can be coupled to the sterile field 80 and can provide an easily accessible anchor point to selectively couple the device 200 thereto. For example, a portion of the sterile field 80, such as a drape or a gown pulled flat may not be easily grasped by the clinician, especially when the clinician is wearing gloves. This can be challenging to then place a portion of the sterile field 80 between the gripping features 228A, 228B when the device is in the open position.

In an embodiment, as shown in FIG. 7A, the anchor 250 can include a first foot plate portion 252A coupled to a first tab portion 260A, and a second tab portion 260B coupled to a second foot plate portion 252B. The first tab portion 260A can be coupled to the second tab portion 260B along a first edge 270 and can be folded there along such that a first side of the first tab portion 260A is disposed adjacent to a first side of the second tab portion 260B. In an embodiment, one or both of the first side of the first tab portion 260A and the first side of the second tab portion 260B can include an adhesive layer disposed thereon and can attach the first side of the first tab portion 260A to the first side of the second tab portion 260B.

In an embodiment, the first foot plate portion 252A can be folded relative to the first tab portion 260A along a second edge 272 and in an opposite direction from the fold along the first edge 270. Similarly, the second foot plate portion 252B can be folded relative to the second tab portion 260B along a third edge 274 and in an opposite direction from the fold along the first edge 270. The second edge 272 and the third edge 274 can be disposed equidistant from the first edge 270. As such, the first surface 254A of the first foot plate portion 252A and the first surface 254B of the second foot plate portion 252A can be aligned along a first axis 64 and the tab 260 can be aligned along a second axis 66 extending at an angle to the first axis 64.

FIGS. 7B-7C show an embodiment, of the anchor 250. FIG. 7B shows the anchor 250 in a first configuration. FIG. 7C shows the anchor 250 in a second configuration. In an embodiment, the anchor 250 can include a foot plate 252 extending over a first plane, e.g. a horizontal plane, and can include an adhesive layer disposed on at least a portion of a first surface 254, e.g. a bottom surface. The anchor 250 can further include a cut line 262 such as a laser cut line, perforation, tear line, or the like. The cut line 262 can define a “U”-shape, “V” shape, or the like extending between a first point 264 and a second point 266 to define the tab 260. The tab 260 can be coupled to the foot plate 252 along a fold line 274 extending along a straight line between the first point 264 and the second point 266.

In an embodiment, the tab 260 can further include a second fold line 278 disposed at a mid-point along the tab 260. As such, the tab 260 can be folded into a first tab portion 260A and a second tab portion 260B so that the second tab portion 260B can support the first tab portion 260A to extend along a second axis 66. One or both of the first fold line 276 and the second fold line 278 can include a score line, groove, laser cut line, perforation, or the like to facilitate folding therealong.

FIG. 7D shows the catheter containment system 198 in an exemplary environment of use. The device 200 is coupled to a proximal portion of the catheter placement system 100 and is secured to an anchor 250. The anchor 250 is adhered to a drape within the sterile field 80, adjacent an insertion site 50. Advantageously, the catheter containment system 198 can secure the proximal portions of the catheter placement system 100 within the sterile field 80 and without affecting the dexterity of the clinician or obstructing the field of view of the insertion site 50 during vascular access and catheter placement.

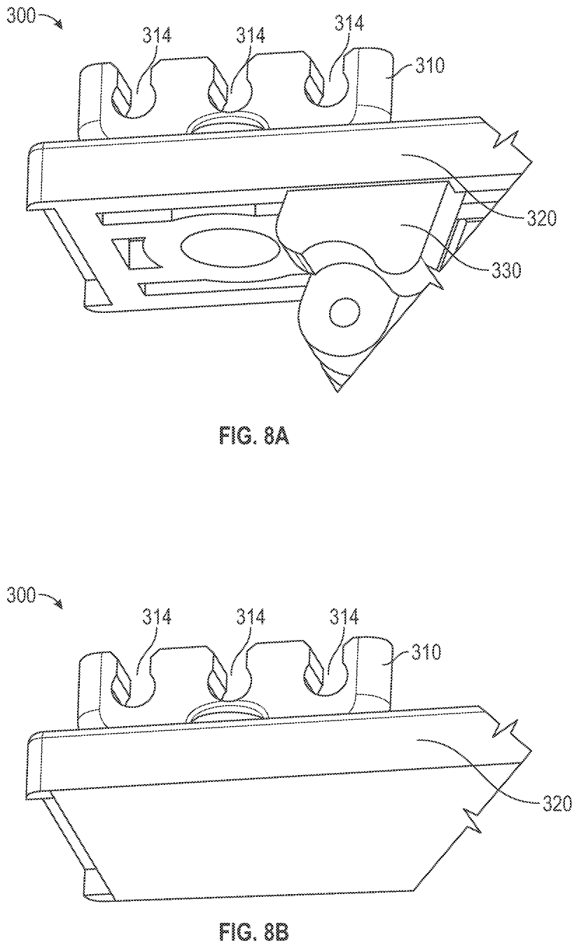

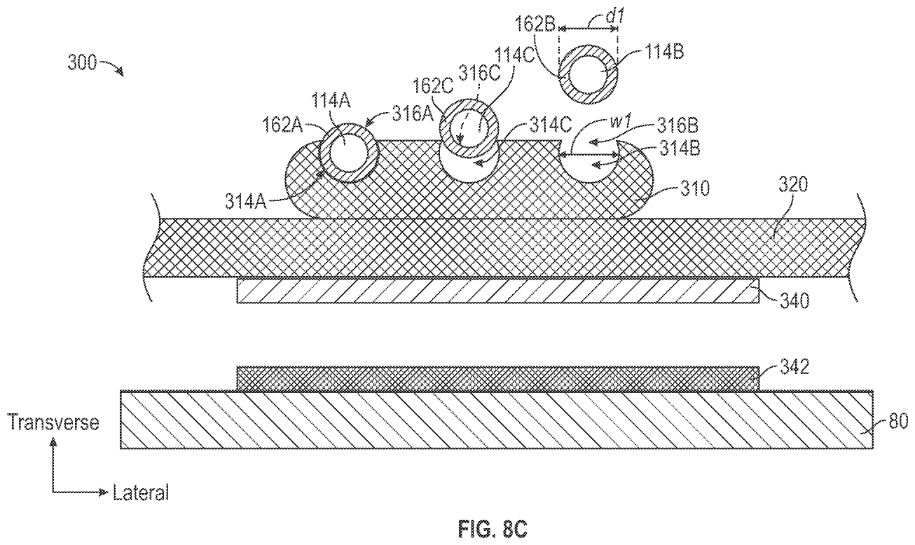

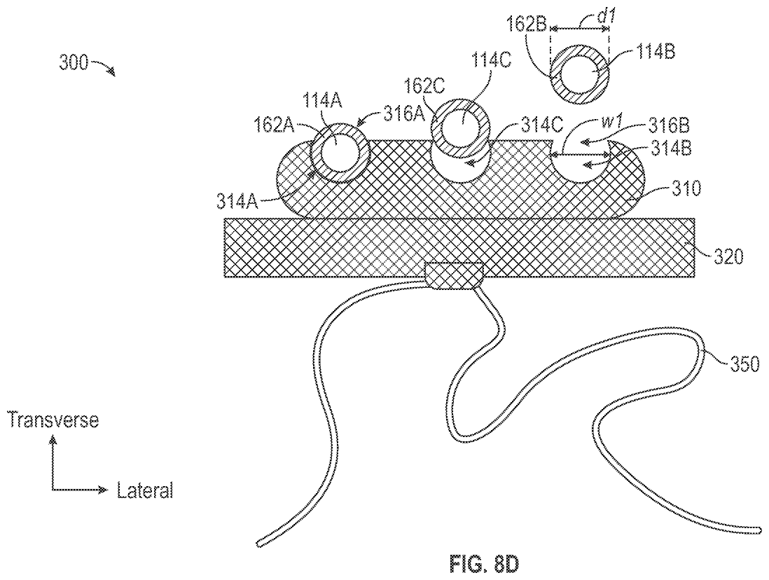

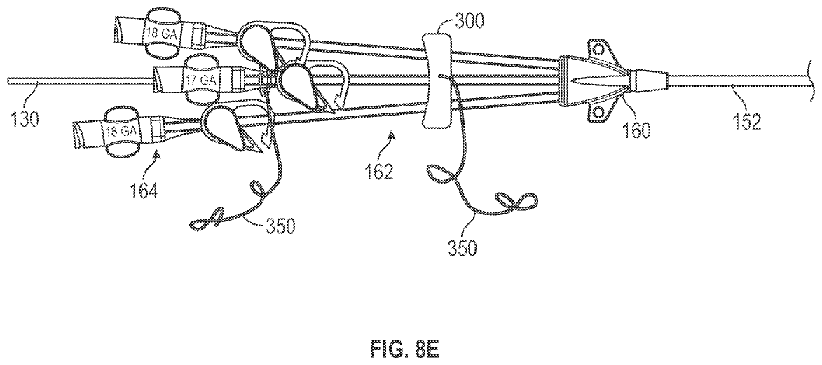

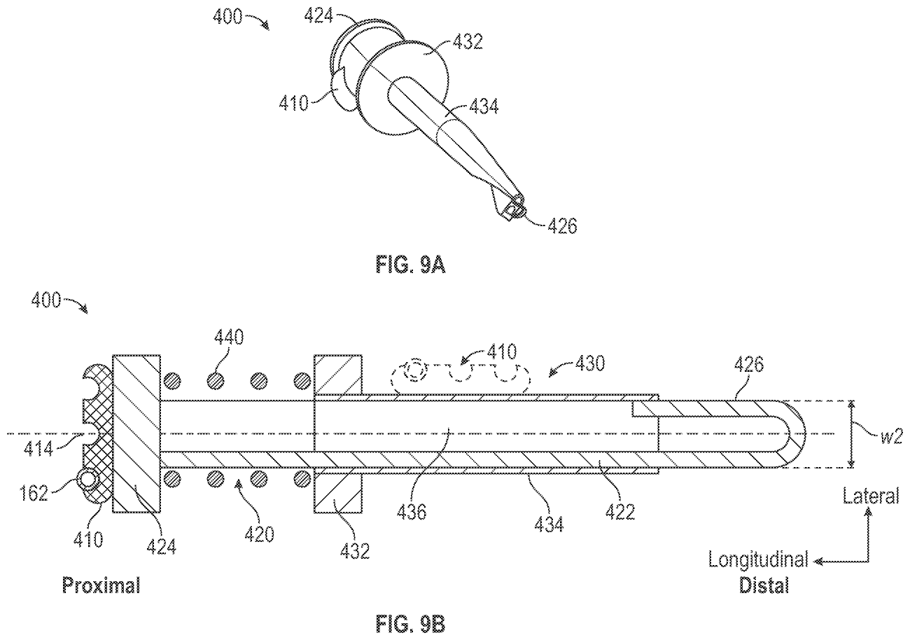

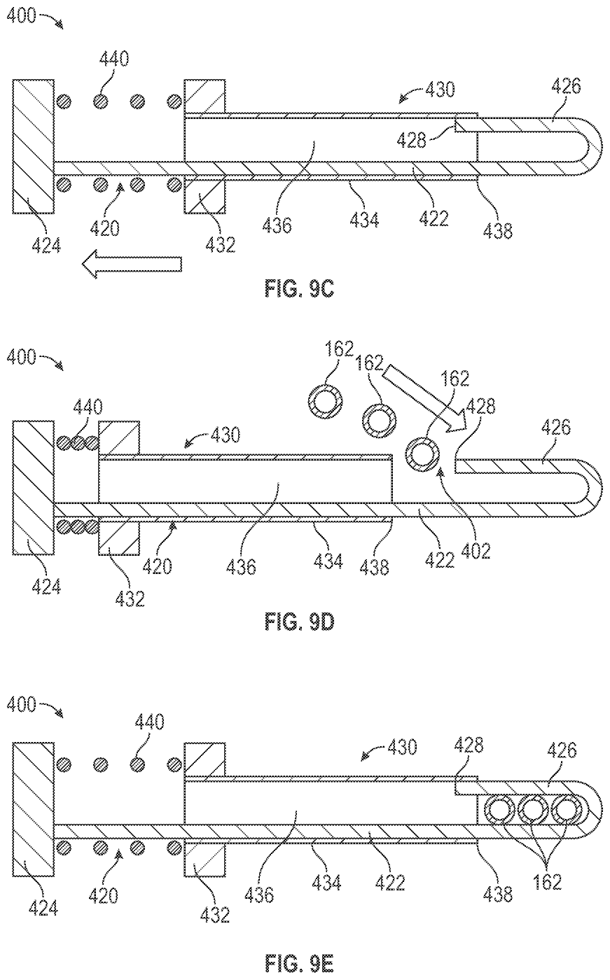

FIGS. 8A-8B show an embodiment of a catheter containment device 300 including a body 310 defining one or more channels 314 and coupled to a base 320. In an embodiment, the channel(s) 314 can be configured to receive one or more portions of the catheter placement system 100, e.g. extension leg 162, or the like, as described herein. In an embodiment, the body 310 can releasably retain, permanently retain, or be permanently coupled to a proximal portion of the catheter placement system 100, e.g. an extension leg 162, guidewire 130, or the like, as described herein. In an embodiment, the body 310 and the base 320 can be formed integrally as a single monolithic piece. In an embodiment, the body 310 and the base 320 can be formed as separate structures and coupled thereto using adhesive, bonding, welding, ultrasonic welding, or the like. In an embodiment, the body 310 and the base 320 can be formed of the same material or of different materials providing different mechanical properties. In an embodiment, the body 310 and the base 320 can be rotatably, pivotably, or hingedly coupled thereto.

In an embodiment, the base 320 can include one or more channels, latches, clips, pinions, or the like configured to releasably engage one or more supporting structures 330 in one or more of an interference fit, press-fit, or snap fit engagement. The support structures 330 can include footings, brackets, clamps, or similar structures configured to be attached to one or more pieces of equipment within the sterile field such as tables, trolleys, beds, gurneys, posts, IV bag hangers, clamps, or the like. In an embodiment, one or more of the base 320 or supporting structures 330 can be pivotably or rotatably coupled to allow the clinician to position the body 310 and the channels 314 in an ergonomically convenient position.

In an embodiment, as shown in FIGS. 8B-8C, the base 320 can define a substantially flat surface. In an embodiment, a first surface of the base 320, e.g. a lower surface, can include an adhesive layer configured to adhere the device 300 to a surface of the sterile field 80. In an embodiment, a first surface of the base 320, e.g. a lower surface, can include an a second material having a relatively high frictional co-efficient. Exemplary materials can include rubber, silicone rubber, polymers, elastomers, or the like and can provide a “non-slip” surface to engage a portion of the sterile field 80.

In an embodiment, a first surface of the base 320, e.g. a lower surface, can include a first coupling 340. A surface of the sterile field 80 can include a second coupling 342 configured to releasably engage the first coupling 340. To note, the body 310 can be coupled to a second surface of the base 320. In an embodiment, the second surface of the base 320 can be opposite the first surface. It will be appreciated that the body 310 can be coupled to any surface of the base 320 without limitation.

In an embodiment, the first coupling 340 can be formed integrally with the base 320. In an embodiment, the first coupling 340 can be formed as a separate structure and coupled with the base 320 using adhesive, bonding, welding, ultrasonic welding, or the like. In an embodiment, the second coupling 342 can be formed integrally with the sterile field 80. In an embodiment, the second coupling 342 can be formed as a separate structure and coupled with the sterile field 90 using adhesive, bonding, welding, ultrasonic welding, or the like.

In an embodiment, the first coupling 340 and the second coupling 342 can be a hook and loop, or VELCRO®, system. For example, the first coupling 340 can be a hook system configured to releasably engage a loop system formed integrally, or adhered to, a surface of the sterile field 80. As will be appreciated, the second coupling 342 can be a hook system configured to releasably engage a loop system formed integrally, or adhered to, a surface of the base 320.

In an embodiment, the first coupling 340 and the second coupling 342 can be a snap fastener system. For example, the first coupling 340 can be a male snap fastener system configured to releasably engage a female snap fastener system of the second coupling 342 formed integrally, or coupled with, a surface of the sterile field 80. As will be appreciated, the second coupling 342 can be a male snap fastener system configured to releasably engage a female snap fastener system formed integrally, or adhered to, a surface of the base 320.

In an embodiment, one or both of the first coupling 340 and the second coupling 342 can be a magnetic element configured to releasably engage the base 320 with the sterile field 80. As used herein, a magnetic element can include a permanent magnet, electro-magnet, a magnetized ferrous material, or a ferrous material. In an embodiment, the magnetic element second coupling 342 can be disposed outside of the sterile field. In an embodiment, the magnetic element second coupling 342 can retain a portion of the sterile field 80 between the magnetic first coupling 340 and the magnetic second coupling 342 to secure the catheter containment device 300 thereto.

In an embodiment, one or both of the first coupling 340 and the second coupling 342 can be a permanent or temporary adhesive configured to couple the base 320 with the sterile field 80. In an embodiment, the first coupling 340 can engage a proximal portion of the catheter placement system 100 directly and can engage the second coupling 342 coupled with the sterile field 80 to secure the proximal portion of the catheter placement system 100 directly thereto.

In an embodiment, as shown in FIG. 8D, the base can include a tie 350 coupled thereto and configured to tie the base 320 to a portion of the sterile field 80, for example a bar, post, I.V. bag hanger, bed, gurney, portion of a drape, sleeve, glove, or the like. In an embodiment, the tie 350 can be a woven or none woven, synthetic or organic material such as a string, cord, wire, twist tie, zip tie, and formed of a plastic, polymer, metal, alloy, composite, cotton, nylon, combinations thereof or the like. In an embodiment, as shown in FIG. 8E, the tie 350 can be coupled directly with a proximal portion of the catheter placement system 100 and can couple the proximal portion of the catheter placement system 100 to a portion of the sterile field 80, as described herein.

FIGS. 9A-9B show an embodiment of a grasping catheter containment device (“grasping device”) 400. The grasping device 400 can generally include a hook 420 slidably engaged with a sleeve 430 between an open position and a closed position. The hook 420 can co-operate with the sleeve 430 to releasably retain one of a portion of a catheter placement system 100 or a portion of a sterile field 80. In an embodiment, where the grasping device 400 is configured to releasably retain a portion of the sterile field 80, the grasping device 400 can further include a body 410. The body 410 can be coupled to the grasping device 400, e.g. to the hook 420, the sleeve 430, or the like. The body 410 can include one or more channels 414 disposed therein. The channel 414 can be configured to engage a portion of the catheter placement system 100, such as an extension leg 162, luer lock 164, hub 160, etc. as described herein. In an embodiment, the channel 414 can releasably retain a proximal portion of the catheter placement system 100, e.g. an extension leg 162, guidewire 130, or the like, as described herein. In an embodiment, a proximal portion of the catheter placement system 100, e.g. guidewire 130, plug 132, etc. can be permanently coupled to the device 400, as described herein.

The hook 420 can include an arm 422 extending along an axis from a first finger plate 424 disposed at a first end, to a J-hook 426 disposed at a second end. The J-hook 426 can extend from a tip of the arm 422 at the second end, towards the first end to a tip 428 of the J-hook 426, i.e. to a point that is between the first end and the second end. In an embodiment, the sleeve 430 can include a second finger plate 432 disposed at a first end, and a sleeve body 434 extending toward the second end from the second finger plate 432. The sleeve 430 can define a lumen 436 extending axially. A portion of the hook 420 can be slidably engaged with the sleeve lumen 436 and can transition between an open configuration and a closed configuration. The grasping device 400 can further include a biasing member 440 configured to bias the grasping device 400 to a closed position. In an embodiment, the biasing member 440 can be compression spring, leaf spring, rubber O-ring, or similar device that is elastically deformable, as described herein. In an embodiment, a lateral width (w2) between a radially outer most point of the J-hook 246 and a radially outer most point of the arm 422 can be substantially equal to an inner diameter of the sleeve lumen 436.

FIGS. 9C-9E show an exemplary method of use for a grasping device 400, as described herein. As shown in FIG. 9C the clinician can compress the first finger plate 424 of the hook 420 and the second finger plate 432 of the sleeve 430 towards each other to overcome the force of the biasing member 440 and transition the grasping device 400 from the closed position to the open position. In the closed position, as shown in FIG. 9C, the tip 428 of the J-hook 426, can be disposed within the lumen of the sleeve body 434. In the open position, as shown in FIG. 9D, the tip 428 of the J-hook 426 can be disposed beyond a tip 438 of the sleeve body 434 and can provide an opening 402 therebetween.

In an embodiment, a proximal portion of the catheter placement system 100, for example one or more extension legs 162 can be disposed through the opening 402 and can be received between the J-hook 426 and the arm 422. Advantageously, securing one or more extension legs 162 together facilitates speed of placement while preventing contamination. In an embodiment, a portion of the sterile field 80, can be disposed through the opening 402 and can be received between the J-hook 426 and the arm 422. As shown in FIG. 9E, the clinician can then release one or both of the first finger plate 424 and the second finger plate 432 to allow the biasing member 440 to transition the grasping device 400 from the open position to the closed position and retain the proximal portion of the catheter placement system 100, or the portion of the sterile field 80, within the hook 420.

In an embodiment, with the portion of the catheter placement system 100 received by the hook 420, the grasping device 400 can be secured to a portion of the sterile field 80 by way of temporary or permanent adhesive, a spring clip 220, an anchor 250, a base 320, a support structure 330, a first coupling 340 and a second coupling 342, a tie 350, combinations thereof, or the like. In an embodiment, the grasping device 400 can be formed integrally with a portion of the sterile field 80, e.g. a portion of a drape or gown worn by one of the clinician or the patient.

In an exemplary method of use, the grasping device 400 can include a body 410 having one or more channels 414. The clinician can urge a proximal portion of the catheter placement system 100 into the channel 414 to be retained therein, as described herein. The body 410 can be coupled to the grasping device 400, for example to one of the hook 420 or the sleeve 430. The grasping device 400 can be transitioned from a closed position to an open position by urging the first finger plate 424 and the second finger plate 432 towards each other, as described herein. A portion of the sterile field 80 can then be placed through the opening 402 and placed between the J-hook 426 and the arm 422. The clinician can then release one or both of the first finger plate 424 and the second finger plate 432 to allow the biasing member 440 to transition the grasping device 400 from the open position to the closed position and retain the portion of the sterile field 80 within the hook 420. The grasping device 400 can then retain the proximal portion of the catheter placement system 100 within the sterile field 80.

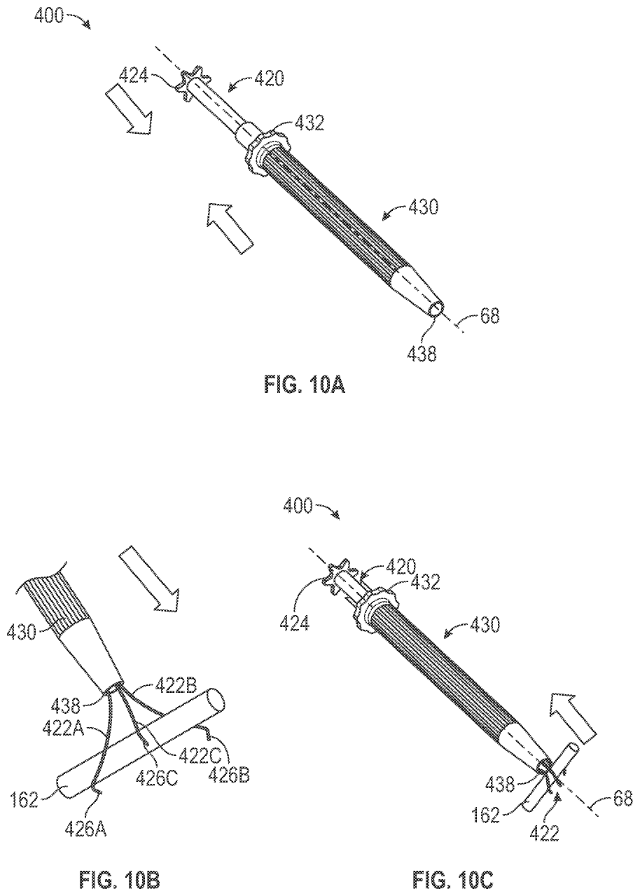

In an embodiment, as shown in FIGS. 10A-10C, the grasping device 400 can include two or more arms 422. As shown, the grasping device 400 can include three arms 422, i.e. a first arm 422A, a second arm 422B, and a third arm 422C. However, greater or lesser numbers of arms 422 are contemplated. In an embodiment, the arm 422 can be formed of a resilient, rigid, or elastically deformable material such as a plastic, polymer, metal, alloy, composite, or the like. In an embodiment, one or more of the first arm 422A, second arm 422B, and third arm 422C can include a J-hook 426, e.g. a first J-hook 426A, a second J-hook 426B, and a third J-hook 426C disposed at a distal tip of the first arm 422A, second arm 422B, and third arm 422C, respectively.

In an embodiment, the arms 422 can be slidable relative to the sleeve 430 between a retracted position (FIG. 10A), an open position (FIG. 10B), and a closed position (FIG. 10C). In the open position, as shown in FIG. 10B, the arms 422 can be disposed in an extended position relative to the sleeve 430. In the closed position, as shown in FIG. 10C, the arms 422 can be disposed in relatively retracted position, relative to the open position. In a retracted position, as shown in FIG. 10A, the arms 422 can be retracted within the sleeve 430. In an embodiment, a distal portion of the arm 422 can be curved radially outwards from a central axis 68 and can be elastically deformed to a relatively less curved shape, or relatively more linear shape, as the arms 422 are retracted into the sleeve 430.

In an exemplary method of use, the grasping device 400 including two or more arms 422 is provided, as described herein. As shown in FIG. 10A, in a resting state, a biasing member 440 can bias the arms 422 to a retracted position where the arms 422 can be disposed within the sleeve lumen 436 and can be elastically deformed to a relatively less curved, or substantially linear shape.

The clinician can then transition the grasping device 400 from the retracted position to an open position (FIG. 10B). The clinician can urge the first finger plate 424 and the second finger plate 432 towards each other to overcome the force of the biasing member 440 and advance the tips of the arms 422 to extend beyond a tip 438 of the sleeve. In an embodiment, the arms 422 in the open position can grasp a portion of the catheter placement system 100, e.g. an extension leg 162. In an embodiment, the arms 422 in the open position can grasp a portion of the sterile field 80.

The clinician can then release one or both of the first finger plate 424 and the second finger plate 432 to allow the biasing member 440 to transition the arms to the closed position (FIG. 10C). In the closed position the arms 422 are extending from the sleeve lumen 436 but are in a relatively more retracted position relative to the open position (FIG. 10B). As such the arms 422 can close about the proximal portion of the catheter placement system 100 or the sterile field 80.

In an embodiment, as shown in FIG. 11, the catheter containment device 500 can include a securement feature such as one or more of a spring clip 220, butterfly clip 220A, beret clip 220B, alligator clip 220C, or a clothes pin 220D, as described herein and configured to engage both a proximal portion of the catheter placement system 100 and a portion of the sterile field 80 between the first gripping feature 228A and the second gripping feature 228B, securing the proximal portion of the catheter placement system 100 within the sterile field 80.

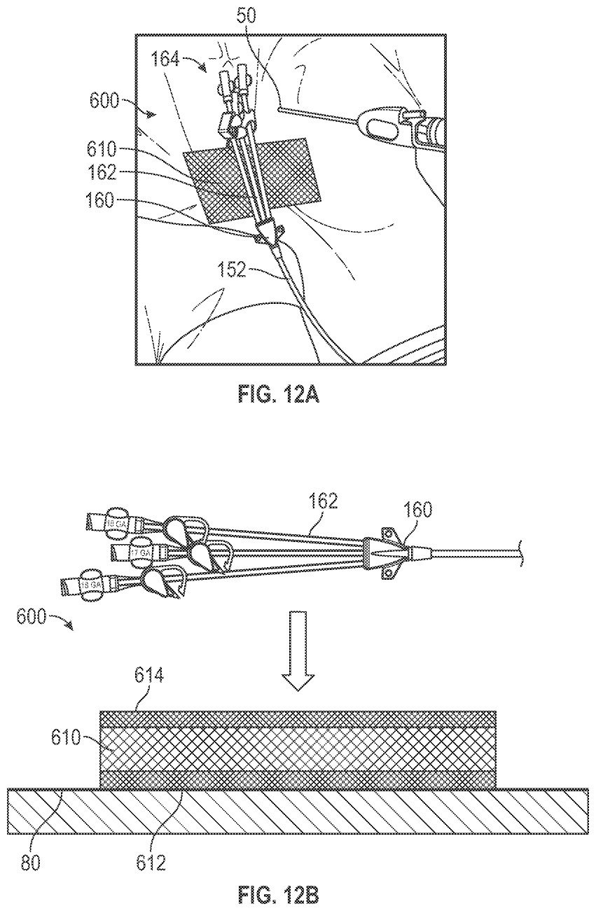

As shown in FIGS. 12A-12B, in an embodiment, the catheter containment device 600 can include a pad 610 including a material having a high frictional co-efficient and coupled to a portion of the sterile field 80. The pad 610 can include a first surface that engages a surface of the sterile field 80, and a second surface 614 opposite the first surface and including a high friction co-efficient material. In an embodiment, the pad 610 can be formed of a rigid or resilient material. In an embodiment, the pad 610 can be formed of a flexible, malleable, or elastically deformable material. In an embodiment, the pad 610 can be formed of a first material having rigid or resilient mechanical properties, and can include a second material disposed thereon, the second material displaying softer, more flexible mechanical properties, relative to the first material. The second material can display a higher frictional co-efficient than the first material. In an embodiment, one or both of the first material and the second material can include a plastic, polymer, elastomer, rubber, silicone rubber, metal, alloy, composite, or the like. The second surface 614 can include a material having a high friction coefficient such as silicone rubber, rubber, latex, or the like. In an embodiment, the second surface 614 of the pad 610 can include a coating having a high friction coefficient. In an embodiment, the second surface 614 of the pad 610 can include a relatively light tack or temporary adhesive coating.

In an embodiment, the pad 610 can include an indicator such as a different color, opaque surface, symbol, or alphanumeric symbol, combinations thereof, or the like, to indicate the location of the catheter containment pad 610 within the sterile field 80. In an embodiment, the pad 610 can be formed integrally with the sterile field 80, e.g. formed integrally with a drape or surgical gown. In an embodiment, the pad 610 can be releasably coupled with the sterile field 80, with a snap fastener, hook and loop attachment, adhesive, or the like disposed on the first surface. In an embodiment, the pad 610 can be coupled with the sterile field 80 using a permanent adhesive.

In an exemplary method of use, the pad 610 can be provided, as described herein. A first surface of the pad 610 can engage the surface of the sterile field 80. A clinician can place a proximal portion of the catheter placement system 100 on the second surface 614 of the pad 610. The high-frictional co-efficient of the second surface, or the adhesive, can retain the proximal portion of the catheter placement system 100 thereon preventing the proximal portion of the catheter placement system 100 from falling outside of the sterile field 80.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter containment system for retaining a portion of a catheter placement system within a sterile field, comprising:
a containment device comprising:
a body defining a channel extending longitudinally along a first axis, the channel configured to retain the portion of the catheter placement system, and having an opening extending longitudinally and configured to allow ingress or egress along a second axis extending at an angle to the first axis of the channel; and
a securement feature coupled to the body and configured to releasably engage a portion of the sterile field proximate an insertion site.

2. The catheter containment system according to claim 1, wherein the portion of the catheter placement system includes one of a catheter body, catheter hub, extension leg, luer lock, or guidewire.

3. The catheter containment system according to claim 2, wherein the body includes a first channel configured to retain a first extension leg and a second channel configured to retain a second extension leg.

4. The catheter containment system according to claim 2, wherein the body includes a first channel configured to retain a first extension leg and a second extension leg.

5. The catheter containment system according to claim 1, wherein the catheter placement system includes a RICC catheter placement system for placing a rapidly insertable central catheter.

6. The catheter containment system according to claim 1, wherein the body is rotatably, hingedly or pivotably coupled to the securement feature.

7. The catheter containment system according to claim 1, wherein the securement feature includes one of a spring clip, butterfly clip, beret clip, alligator clip, clothes pin, safety pin, or suction cup.

8. The catheter containment system according to claim 1, further including an anchor having a foot plate and a tab extending at an angle thereto, the foot plate include an adhesive layer and the tab configured to be engaged by the securement feature.

9. The catheter containment system according to claim 1, wherein the securement feature includes a first clip member hingedly coupled to a second clip member, the first clip member including a first gripping feature configured to engage a second gripping feature of the second clip member to secure the containment device to the portion of the sterile field.

10. The catheter containment system according to claim 1, wherein the securement feature further includes a biasing member configured to bias the securement feature to a closed position, the biasing member including one of a spring, compression spring, leaf spring, O-ring, rubber grommet, or magnetic element.

11. The catheter containment system according to claim 1, further including a base coupled to both the body and the securement feature, and wherein the securement feature includes a first coupling disposed on the base, and a second coupling disposed on the portion of the sterile field.

12. The catheter containment system according to claim 11, wherein the securement feature includes one of a hook and loop fastener, snap fastener, a magnetic element, or adhesive.

13. The catheter containment system according to claim 1, wherein the securement feature includes a tie selected from a group consisting of a string, cord, wire, twist tie, or zip tie.

14. The catheter containment system according to claim 1, wherein the securement feature includes a hook slidably engaged with a sleeve, the hook having an arm with a finger plate disposed at a first end of the arm and a J-hook disposed at a second end of the arm, opposite the finger plate, the arm configured to receive a portion of the sterile field between the J-hook and the sleeve.

15. The catheter containment system according to claim 1, wherein the securement feature includes a sleeve and two or more hooks slidably engaged therewith, each of the two or more hooks slidable between a retracted position, open position, and a closed position.

16. A catheter placement and containment system for placing a catheter within a vasculature of a patient, comprising:
a catheter placement device, comprising:
a needle;
a catheter including an extension leg; and
a guidewire;
a sterile drape configured to define a sterile field around the insertion site; and
a catheter containment system, comprising:
a body defining a channel and configured to engage a portion of the catheter placement device; and
a securement feature coupled to the body and configured to secure the catheter containment system to the sterile drape.

17. The system according to claim 16, wherein the securement feature includes one of a spring clip, butterfly clip, beret clip, alligator clip, clothes pin, safety pin, or suction cup.

18. The system according to claim 16, further including an anchor having a foot plate and a tab extending at an angle thereto, the foot plate include an adhesive layer and the tab configured to be engaged by the securement feature.

19. The system according to claim 16, further including a base coupled to both the body and the securement feature, and wherein the securement feature includes a first coupling disposed on the base, and a second coupling disposed on the sterile drape.

20. The system according to claim 19, wherein the securement feature includes one of a hook and loop fastener, snap fastener, a magnetic element, or adhesive.

21. A method of placing a catheter, comprising:
providing a catheter placement system including a needle, a catheter, and a guidewire;
releasably engaging a portion of the catheter placement system with the catheter containment device;
securing the catheter containment device with a portion of a sterile field; and
placing the catheter within a vasculature through an insertion site within the sterile field.

22. The method according to claim 21, further including adhering the portion of the catheter placement system to a top surface of the catheter containment device, the catheter containment device comprising a flexible pad coupled to the portion of the sterile field.

23. The method according to claim 21, wherein the catheter containment device includes one of a spring clip, butterfly clip, beret clip, alligator clip, clothes pin, safety pin, suction cup, string, cord, wire, twist tie, or zip tie.

24. A catheter containment device for retaining a portion of a catheter placement system, comprising:
a body defining a channel extending along a longitudinal axis, the channel configured to permanently retain the portion of the catheter placement system; and
a handle coupled to the body and configured to releasably engage a portion of a sterile field proximate an insertion site.

25. The catheter containment device according to claim 24, wherein the portion of the catheter placement system includes a proximal end of a guidewire permanently affixed to the body.

26. The catheter containment device according to claim 24, wherein the portion of the catheter placement system includes a proximal portion of a guidewire slidably engaged with the body.
